(19) United States
(12) Patent Application Publication
(10) Pub. No.: US 2024/0298912 A1
(43) Pub. Date: Sep. 12, 2024

(54) MAGNETIC RESONANCE SCANNING AND IMAGING METHOD AND MAGNETIC RESONANCE IMAGING SYSTEM

(71) Applicant: GE Precision Healthcare LLC, Waukesha, WI (US)

(72) Inventors: Ting Zhang, Beijing (CN); Yongchuan Lai, Beijing (CN); Wei Chang, Beijing (CN); Teresa Weng, Beijing (CN); Jia Guo, Beijing (CN)

(21) Appl. No.: 18/597,760

(22) Filed: Mar. 6, 2024

(30) Foreign Application Priority Data
Mar. 7, 2023 (CN) 202310214670.2

(51) Int. Cl. A61B 5/026; G01R 33/54; G01R 33/563

(52) U.S. Cl. CPC A61B 5/0263; G01R 33/543; G01R 33/56308

(57) ABSTRACT

A magnetic resonance scanning and imaging method, and a magnetic resonance imaging system is presented. The method includes: according to a first correspondence between a blood flow rate and a spatial saturation band parameter, determining a first spatial saturation band parameter corresponding to a blood flow rate of a site to be examined; and by using a scan sequence related to the first spatial saturation band parameter, scanning the site to be examined, to acquire a magnetic resonance image of the site to be examined.

### CROSS REFERENCE TO RELATED APPLICATIONS

The present application claims priority and benefit of Chinese Patent Application No. 202310214670.2 filed on Mar. 7, 2023, which is incorporated herein by reference in its entirety.

### TECHNICAL FIELD

Embodiments of the present application relate to the technical field of medical devices, and in particular to a magnetic resonance scanning and imaging method and a magnetic resonance imaging system.

### BACKGROUND

Magnetic resonance (MR) imaging systems have been widely applied in the field of medical diagnostics. A magnetic resonance system typically has a main magnet, a gradient amplifier, a radio-frequency amplifier, a gradient coil, a transmit chain module, a transmit/receive coil, a receive chain module, and the like. The transmit chain module generates a pulse signal and transmits same to the transmit/receive coil. The transmit/receive coil generates a radio-frequency excitation signal to excite a scan subject to generate a magnetic resonance signal. After excitation, the transmit/receive coil receives the magnetic resonance signal, and a medical image is reconstructed according to the magnetic resonance signal.

Spatial saturation technology is a commonly used technology in MR, in which a region of interest is selectively excited by applying a saturation pulse, so that the region of interest is saturated under the excitation of a scan pulse (the magnetic resonance signal being weakened or even disappearing due to an insufficient magnetization vector) without generating a signal. A commonly used spatial saturation technique is spatial saturation band technology, which, for example, suppresses a blood flow signal in the region of interest by means of configuring a suitable spatial saturation band parameter, reducing artifacts in the medical image caused by blood flow.

### SUMMARY

The embodiments of the present application provide a magnetic resonance scanning and imaging method and a magnetic resonance imaging system.

According to an aspect of the embodiments of the present application, a magnetic resonance scanning and imaging method is provided, the method comprising:
Determining, according to a first correspondence between a blood flow rate and a spatial saturation band parameter, a first spatial saturation band parameter corresponding to a blood flow rate of a site to be examined; and
By using a scan sequence related to the first spatial saturation band parameter, scanning the site to be examined, to acquire a magnetic resonance image of the site to be examined.

According to an aspect of the embodiments of the present application, a magnetic resonance imaging system is provided, comprising: a scanning unit; and a controller used to determine, according to a first correspondence between a blood flow rate and a spatial saturation band parameter, a first spatial saturation band parameter corresponding to a blood flow rate of a site to be examined; and control the scanning unit to use a scan sequence related to the first spatial saturation band parameter to scan the site to be examined, to acquire a magnetic resonance image of the site to be examined.

One of the benefits of the embodiments of the present application is that the corresponding spatial saturation band parameter is automatically determined according to the blood flow rate of the site to be examined, and the site to be examined is scanned using the scan sequence related to the first spatial saturation band parameter, to acquire the magnetic resonance image of the site to be examined. As such, artifact signals in the magnetic resonance image caused by blood flow may be better suppressed, making the magnetic resonance imaging clearer, and the workload of a user can be reduced, improving work efficiency.

With reference to the following description and drawings, specific implementations of the embodiments of the present application are disclosed in detail, and the means by which the principles of the embodiments of the present application can be employed are illustrated. It should be understood that the implementations of the present application are therefore not limited in scope. Within the scope of the spirit and clauses of the appended claims, the implementations of the present application comprise many changes, modifications, and equivalents.

### BRIEF DESCRIPTION OF THE DRAWINGS

The included drawings are used to provide further understanding of the embodiments of the present application, which constitute a part of the description and are used to illustrate the implementations of the present application and explain the principles of the present application together with textual description. Evidently, the drawings in the following description are merely some embodiments of the present application, and a person of ordinary skill in the art may obtain other implementations according to the drawings without involving inventive skill. In the drawings:

FIG. 1 is a schematic diagram of a magnetic resonance imaging system according to embodiments of the present application;

FIG. 2 is a schematic diagram of a magnetic resonance scanning and imaging method according to embodiments of the present application;

FIGS. 3 and 4 are schematic diagrams of spatial saturation band parameters according to embodiments of the present application;

FIG. 5 is a schematic diagram of a method for determining a first correspondence according to embodiments of the present application;

FIGS. 6 and 7 are schematic diagrams of a plurality of blood flow magnetic resonance signals according to embodiments of the present application;

FIG. 8 is a schematic diagram of a second pulse sequence according to embodiments of the present application;

FIG. 9 is a schematic diagram of a first pulse sequence according to embodiments of the present application;

FIG. 10 is a schematic diagram of a magnetic resonance scanning and imaging method according to embodiments of the present application;

FIG. 11 is a schematic diagram of a method for determining a first correspondence according to embodiments of the present application;

FIG. 12 is a schematic diagram of a magnetic resonance scanning and imaging method according to embodiments of the present application; and

FIG. 13 is a schematic diagram of an apparatus for determining a spatial saturation band parameter according to embodiments of the present invention.

### DETAILED DESCRIPTION

The foregoing and other features of the embodiments of the present application will become apparent from the following description and with reference to the drawings. In the description and drawings, specific implementations of the present application are disclosed in detail, and part of the implementations in which the principles of the embodiments of the present application may be employed are indicated. It should be understood that the present application is not limited to the described implementations. On the contrary, the embodiments of the present application include all modifications, variations, and equivalents which fall within the scope of the appended claims.

In the embodiments of the present application, the terms “first”, “second”, etc., are used to distinguish different elements, but do not represent a spatial arrangement or temporal order, etc., of these elements, and these elements should not be limited by these terms. The term “and/or” includes any and all combinations of one or more associated listed terms. The terms “comprise”, “include”, “have”, etc., refer to the presence of described features, elements, components, or assemblies, but do not exclude the presence or addition of one or more other features, elements, components, or assemblies.

In the embodiments of the present application, the singular forms “a”, “the”, etc., include plural forms, and should be broadly construed as “a type of” or “a class of” rather than limited to the meaning of “one.” Furthermore, the term “said” should be construed as including both the singular and plural forms, unless otherwise specified in the context. In addition, the term “according to” should be construed as “at least in part according to . . . ” and the term “on the basis of” should be construed as “at least in part on the basis of . . . ”, unless otherwise specified in the context.

The features described and/or illustrated for one implementation may be used in one or more other implementations in the same or similar manner, be combined with features in other embodiments, or replace features in other implementations. The terms “include/comprise” when used herein refer to the presence of features, integrated components, steps, or assemblies, but do not preclude the presence or addition of one or more other features, integrated components, steps, or assemblies.

For case of understanding, FIG. 1 shows a magnetic resonance imaging (MRI) system 100 according to some embodiments of the present invention.

The MRI system 100 includes a scanning unit 110. The scanning unit 110 is used to perform a magnetic resonance scan of a subject (e.g., a human body) 16 to generate image data of a region of interest of the subject 16, wherein the region of interest may be a pre-determined anatomical site or anatomical tissue.

The magnetic resonance imaging system 100 may include a controller 130 which is coupled to the scanning unit 110 and indicates an MRI scan sequence to be performed during the MRI scan, so as to control the scanning unit 110 to perform the flow of the aforementioned magnetic resonance scan.

The scanning unit 110 may include a main magnet assembly 111. The main magnet assembly 111 typically includes an annular superconducting magnet defined in a housing. The annular superconducting magnet is mounted in an annular vacuum container. The annular superconducting magnet and the housing thereof define a cylindrical space surrounding the subject 16, such as an imaging space 120 shown in FIG. 1. The main magnet assembly 111 generates a constant magnetic field, i.e., a B0 field, in a Z direction of the imaging space 120.

Usually, the Z direction is typically the direction extending from the head to the feet (or from the feet to the head) when the subject 16 is positioned on a table 112. For example, a selected layer may be a slice at any position in the Z direction. A uniform portion of the B0 field is formed in a central region of the main magnet.

The scanning unit 110 further includes a table 112, which is used to carry the subject 16 and travel, in response to the control of the controller 130, in the Z direction to enter or exit a scanning chamber. For example, in an embodiment, an imaging volume of the subject 16 may be positioned in a central region of the imaging space 120 having uniform magnetic field strength, so as to facilitate scanning and imaging of the imaging volume of the subject 16.

The magnetic resonance imaging system 100 uses the formed B0 field to transmit a static magnetic field to the subject 16 located in the scanning chamber, so that protons in a resonant region in the body of the subject 16 precess in an ordered manner.

The scanning unit 110 further includes a radio frequency driver 113 and a radio-frequency transmit coil 114. The radio-frequency transmit coil 114 is configured, for example, to surround a region to be imaged of the subject 16. The radio-frequency transmit coil 114 may include, for example, a body coil disposed along an inner circumference of the main magnet, or a local coil dedicated to local imaging. The radio frequency driver 113 may include a radio frequency generator (not shown in the figure), a radio frequency power amplifier (not shown in the figure), and a gate modulator (not shown in the figure). The radio frequency driver 113 is configured to drive the radio-frequency transmit coil 114 and form a high-frequency magnetic field in space. Specifically, the radio frequency generator generates a radio-frequency excitation signal on the basis of a control signal from the controller 130. The gate modulator modulates the radio-frequency excitation signal into a signal having a predetermined envelope and predetermined timing. After being amplified by the radio-frequency power amplifier, the modulated radio-frequency excitation signal is outputted to the radio-frequency transmit coil 114, so that the radio-frequency transmit coil 114 transmits, to the subject 16, a radio-frequency field that is orthogonal to the BO field, so as to excite protons in a slice to be imaged to spin. After the radio-frequency excitation pulse ends, a magnetic resonance signal is generated during a process of spin-relaxation of the excited protons returning to the initial magnetization vector.

The aforementioned radio-frequency transmit coil 114 may be connected to a transmit/receive (T/R) switch 119. The transmit/receive switch 119 is controlled so that the radio-frequency transmit coil may be switched between a transmit mode and a receive mode. In the receive mode, the radio-frequency transmit coil may be configured to receive, from the subject 16, a magnetic resonance signal having three-dimensional location information.

The three-dimensional location information of the magnetic resonance signal is generated by means of a gradient system of the MRI system, and this will be described in detail below.

The scanning unit 110 further includes a gradient coil driver 115 and a gradient coil assembly 116. The gradient coil assembly 116, in one aspect, forms a magnetic field gradient (a varying magnetic field) in the imaging space 120 so as to provide three-dimensional location information for the magnetic resonance signal, and in another aspect generates a compensating magnetic field of the B0 field to shim the B0 field.

The gradient coil assembly 116 may include three gradient coil systems, and the three gradient coil systems are configured to generate magnetic field gradients that are oblique to three spatial axes (for example, the X-axis, Y-axis, and Z-axis), respectively, which are perpendicular to one another. The gradient coil driver 115 drives the gradient coil assembly 116 on the basis of a control signal from the controller 130, and therefore generates the gradient magnetic field in the imaging space 120. The gradient coil driver 115 includes gradient amplifiers corresponding to the three gradient coil systems in the aforementioned gradient coil assembly, respectively. For example, the gradient coil driver 115 includes a Gz amplifier configured to drive a gradient in a z direction, a Gy amplifier configured to drive a gradient in a y direction, and a Gx amplifier configured to drive a gradient in an x direction.

More specifically, the gradient coil assembly 116 is configured to apply a magnetic field gradient in a slice selection direction (e.g., the z direction) to vary field strength in the region, so that precession frequencies of protons of imaged tissue in different layers (slices) of the region are different and thus layer selection is performed. Those skilled in the art understand that the layer is any one of a plurality of two-dimensional slices distributed in the Z direction in the three-dimensional imaging volume. When the imaging region is scanned, the radio-frequency transmit coil 114 responds to the aforementioned radio-frequency excitation signal, and then a layer having a precession frequency corresponding to this radio-frequency excitation signal is excited. Further, the gradient coil assembly 116 is configured to separately apply a magnetic field gradient in a phase-encoding direction (e.g., the y direction) and a magnetic field gradient in a frequency-encoding direction (e.g., the x direction), so that magnetic resonance signals of excited layers have different phases and frequencies, thereby achieving phase encoding and frequency encoding.

The scanning unit 110 further includes a surface coil 118 which is usually arranged close to a scanned part (a region of interest) of the subject 16 (for example, covering or disposed on the body surface of the subject 16), and the surface coil 118 is also configured to receive the magnetic resonance signal.

The scanning unit 110 further includes a data acquisition unit 117 configured to acquire the magnetic resonance signal (for example, received by the body coil or the surface coil) in response to a data acquisition control signal of the controller 130. In an embodiment, the data acquisition unit 117 may include, for example, a radio-frequency preamplifier (not shown in the figure), a phase detector (not shown in the figure), and an analog/digital converter (not shown in the figure). The radio frequency preamplifier is configured to amplify the magnetic resonance signal. The phase detector is configured to perform phase detection on the amplified magnetic resonance signal. The analog/digital converter is configured to convert the phase-detected magnetic resonance signal from an analog signal into a digital signal.

The data acquisition unit 117 is further configured to store the digitized magnetic resonance signal (or echo) into a K-space in response to a data storage control signal of the controller 130. The K-space is a space to which raw data of magnetic resonance signals carrying spatial orientation encoding information is populated. The data acquisition unit 117, according to a predetermined data filling method, fills signals having different phase information and frequency information in the corresponding locations in the K-spaced. In an example, the two-dimensional K-space may include a frequency-encoding line and a phase-encoding line. Data acquisition at each level may include multiple signal acquisition cycles (or repetition times TR). Each signal acquisition cycle may correspond to one change in the magnetic field gradient (incrementally or decrementally) in the phase-encoding direction (i.e., one signal acquisition is performed for each phase-encoding gradient applied), and the magnetic resonance signal acquired in each signal acquisition cycle is filled into a frequency-encoding line. Through multiple signal acquisition cycles, multiple frequency-encoding lines having different phase information may be filled, and each acquired magnetic resonance signal has multiple decomposition frequencies.

The magnetic resonance imaging system 100 further includes an image reconstructor 140 configured to perform inverse Fourier transform on data stored in the K-space to reconstruct a three-dimensional image or a series of two-dimensional slice images of the imaging volume of the subject 16. Specifically, the image reconstructor 140 may perform the aforementioned image reconstruction on the basis of communication with the controller 130.

The magnetic resonance imaging system 100 further includes a processor 150. The processor 150 may include an image processor for image processing, and the image processor may perform any required image post-processing on the aforementioned three-dimensional image or any image in an image sequence. The post-processing may be an improvement or adaptive adjustment made to an image in any aspect of contrast, uniformity, sharpness, brightness, artifacts, etc. The processor 150 may further include a waveform processor configured to implement a waveform determination method according to an embodiment of the present invention. For example, the waveform processor generates a waveform on the basis of scanning parameters, performs waveform conversion, uses a converted waveform to determine driving/controlling parameters of the gradient amplifier, etc.

In one embodiment, the controller 130, the image reconstructor 140, and the processor 150 may separately or collectively include a computer processor and a storage medium. Recorded on the storage medium is a program for predetermined data processing to be executed by the computer processor. For example, stored on the storage medium may be a program for performing scan processing (e.g., including waveform design/conversion, etc.), image reconstruction, image processing, etc. The aforementioned storage medium may include, for example, a ROM, a floppy disk, a hard disk, an optical disk, a magnetic disk, a CD-ROM, or a non-volatile memory card.

The magnetic resonance imaging system 100 further includes a display unit 160, and the display unit 160 may be configured to display an operation interface and various data, images, or parameters generated during data acquisition and processing processes.

The magnetic resonance imaging system 100 further an operation console 170, and the operation console 170 may include a user input device, such as a keyboard, a mouse, etc. The controller 130 may communicate with the scanning unit 110, the image reconstruction unit 140, the processor 150, the display unit 160, etc., in response to a control command generated by a user on the basis of the operation console 170 or an operation panel/button, etc., disposed on the housing of the main magnet.

Those skilled in the art can understand that when imaging scanning is performed on the subject 16, the controller 130 can send sequence control signals to the aforementioned components (e.g., the radio frequency driver 113, the gradient coil driver 115, etc.) of the scanning unit 110 by means of a sequence generator (not shown in the figure), so that the scanning unit 110 performs a preset scan sequence.

Those skilled in the art can understand that the “scan sequence” (hereafter also referred to as imaging sequence or pulse sequence) refers to a combination of pulses having specific amplitudes, widths, directions, and timings and applied when a magnetic resonance imaging scan is performed. The pulses may typically include, for example, a radio-frequency pulse and a gradient pulse. The radio-frequency pulse may include, for example, a radio-frequency transmission pulse, a radio-frequency refocus pulse, an inverse recovery pulse, etc. The gradient pulse may include, for example, the aforementioned gradient pulse for layer selection, gradient pulse for phase encoding, gradient pulse for frequency encoding, phase balance pulse for phase balancing of proton precession, etc. Typically, a plurality of scanning sequences can be pre-set in the magnetic resonance imaging system, so that the sequence suitable for clinical examination requirements can be selected. The clinical examination requirements may include, for example, an imaging site, an imaging function, an imaging effect, etc.

Currently, a conventional method for configuring a spatial saturation band parameter is manual setting by a physician. However, this operational method may require multiple adjustments, which requires a certain amount of physician experience, increases the workload of the physician, and reduces work efficiency. Even so, configuration of a suitable spatial saturation band parameter cannot be guaranteed, and thus, it can not be guaranteed that the artifacts in the medical image due to blood flow are reduced to the maximum extent.

For at least one of the above problems, in the embodiments of the present application, a corresponding spatial saturation band parameter is automatically determined according to a blood flow rate of a site to be examined, and the site to be examined is scanned using a scan sequence related to a first spatial saturation band parameter, to acquire a magnetic resonance image of the site to be examined. As such, artifact signals in the magnetic resonance image due to blood flow may be better suppressed, making the magnetic resonance imaging clearer, and the workload of a user can be reduced, improving work efficiency.

Description is made below in conjunction with the embodiments.

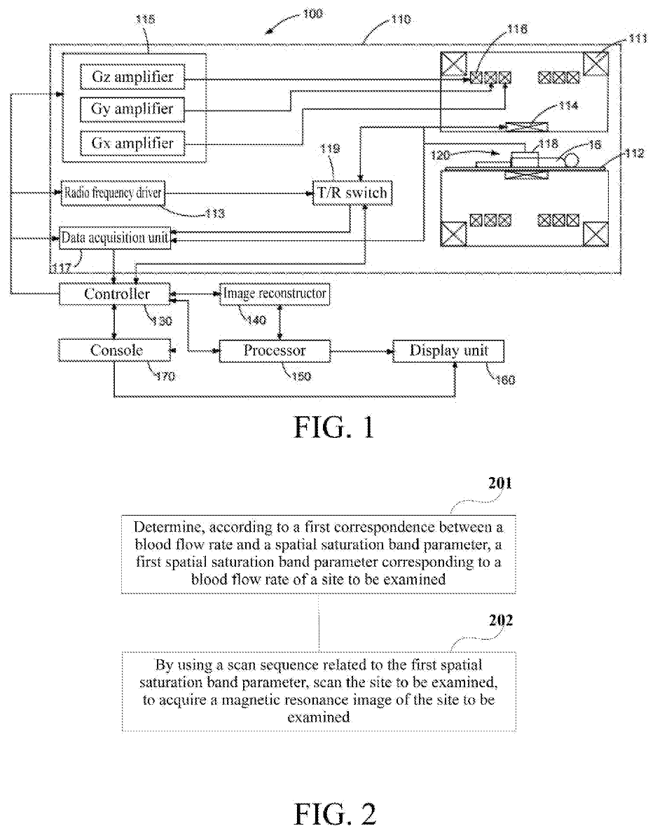

The embodiments of the present application provide a magnetic resonance scanning and imaging method. FIG. 2 is a schematic diagram of the magnetic resonance scanning and imaging method according to the embodiments of the present application.

As shown in FIG. 2, the method includes at step 201, determining, according to a first correspondence between a blood flow rate and a spatial saturation band parameter, a first spatial saturation band parameter corresponding to a blood flow rate of a site to be examined.

At step 202, the method includes, by using a scan sequence related to the first spatial saturation band parameter, scanning the site to be examined, to acquire a magnetic resonance image of the site to be examined.

In some embodiments, blood vessels in different examined sites of the body of the scan subject include at least one of arterial blood vessels and venous blood vessels. In the vessels, the linear velocity at which particles in the blood move is referred to as the blood flow rate. All of the different examined sites have their corresponding blood flow rates. That is, all of the different examined sites have at least one of a corresponding venous blood flow rate and arterial blood flow rate. Therefore, in order to determine the blood flow rate of the site to be examined, the method may include: at step 200, determining a second correspondence between a different examined site and a blood flow rate.

In some embodiments, in 200, at least one of the venous blood flow rates and arterial blood flow rates of different examined sites of different scan subjects may be measured in advance. The measurement method may include a Doppler method, a magnetic resonance method, an injection tracking method, etc. For details, reference may be made to the related art, and the embodiments of the present application are not limited thereto. By collecting the measurement results of the blood flow rates of the different examined sites of the different scan subjects, a second correspondence between a different examined site and a blood flow rate is established. In the second correspondence, the blood flow rate corresponding to the examined site may be a single value (e.g., the average of the measurement results of the different scan subjects for one examined site), or a set of multiple values (which is also referred to as an interval value, for example, the maximum and minimum values of the interval are the maximum and minimum values of the measurement results of the different scan subjects for one examined site). For example, the arterial blood flow rate corresponding to the neck is 100 cm/s-120 cm/s, the venous blood flow rate corresponding to the neck is 5 cm/s-10 cm/s, the arterial blood flow rate corresponding to the abdomen is 100 cm/s-180 cm/s, the venous blood flow rate corresponding to the abdomen is 5 cm/s-20 cm/s, and so on, examples of which are no longer listed herein one by one.

In some embodiments, the spatial saturation band refers to a region (having a long strip shape) in which a radio-frequency pulse having a certain energy is applied in space such that a signal of the tissue is reduced or is zero when the signal is excited and acquired. Setting and adding the saturation band at the time of scanning may be understood to be the addition of a first pulse sequence in the scan sequence. The saturation band refers to a saturation band parallel to a scanning layer or group of layers, and the saturation band may be added on one side or both layers of the scanning layer or group of layers.

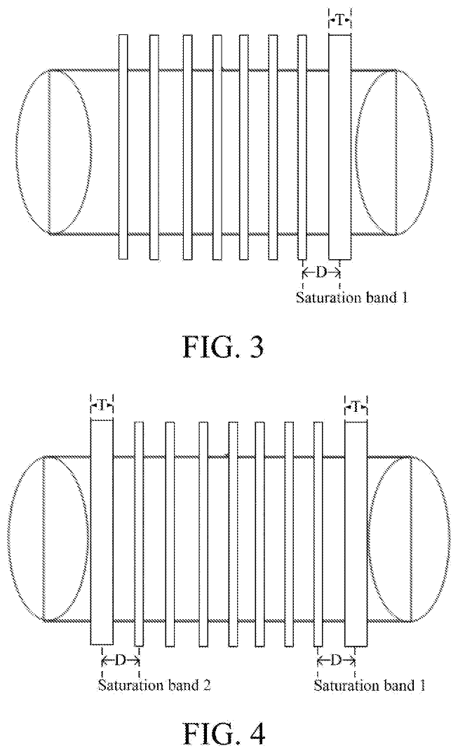

FIGS. 3 and 4 are schematic views of spatial saturation bands according to embodiments of the present application. As shown in FIG. 3, a saturation band 1 is applied on one side of a scanning layer or group of layers. As shown in FIG. 4, a saturation band 1 and a saturation band 2 are applied on both sides of the scanning layer or group of layers. The spatial saturation band parameter determines a parameter of the first pulse sequence applied at the time of scanning.

For example, the spatial saturation band parameter includes at least one of the distance between the spatial saturation band and the site to be examined (the closest scanning layer or group of layers) (GAP) and the thickness of the spatial saturation band (Thickness). As shown in FIGS. 3 and 4, the thickness of the saturation band (Thickness) determines the gradient size of a gradient pulse in the first pulse sequence, a different thickness corresponds to the gradient (Gz) size of a gradient pulse in a different first pulse sequence (for a given radio-frequency pulse bandwidth), and the thickness T is typically set to 5 mm-240 mm. The distance (GAP) determines the center frequency of a radio-frequency pulse in the first pulse sequence, a different distance corresponds to the magnitude of the center frequency of a radio-frequency pulse in a different first pulse sequence, and the distance D is typically set to 0 mm 50 mm. If the distance is too large, it means that the saturation band is too far away from the scanning layer, which may cause a saturated signal not to enter the scanning layer and fail to achieve a saturation effect. If the distance is too small, it means that the saturation band is too close to the scanning layer, which may cause signals in an edge layer to be incorrectly saturated. The above description is merely illustrative. The spatial saturation band parameter may also include other types of parameters, and the embodiments of the present application are not limited thereto. In addition, the first pulse sequence will be described in more detail below.

In some embodiments, when the blood flow rate is different, the blood flow signal (the magnetic resonance blood flow signal) obtained after the magnetic resonance scan is also different, and therefore, the magnitude of the artifact signal generated in the magnetic resonance image due to the blood flow is also different. Therefore, the blood flow signal may be suppressed by setting the spatial saturation band parameter, thus reducing the artifacts in the image. Thus, in order to eliminate artifact signals of different magnitudes, there needs to be a distinction between the set spatial saturation band parameters as well. In the embodiments of the present application, the first correspondence between different blood flow rates and spatial saturation band parameters may be predetermined.

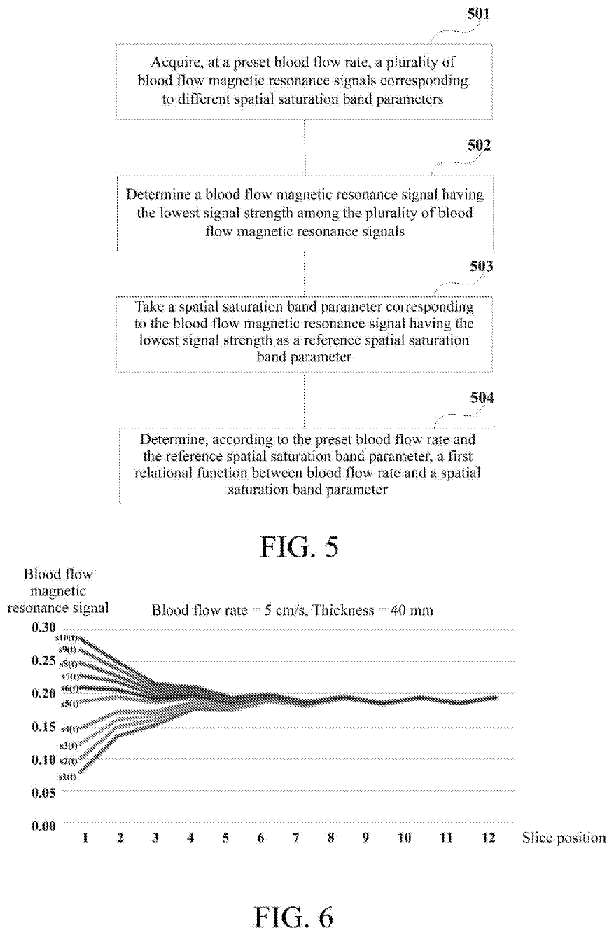

FIG. 5 is a schematic diagram of a method for determining the first correspondence according to embodiments of the present application. As shown in FIG. 5, the method includes: at step 501, Acquiring, at a preset blood flow rate, a plurality of blood flow magnetic resonance signals corresponding to different spatial saturation band parameters.

At step 502, the method includes determining a blood flow magnetic resonance signal having the lowest signal strength among the plurality of blood flow magnetic resonance signals.

Further, at step 503, the method includes taking a spatial saturation band parameter corresponding to the blood flow magnetic resonance signal having the lowest signal strength as a reference spatial saturation band parameter.

At step 504, the method includes determining, according to the preset blood flow rate and the reference spatial saturation band parameter, a first relational function between the blood flow rate and the spatial saturation band parameter.

In some embodiments, 501-504 are each performed for different types of spatial saturation band parameters. For example, for the thickness, 501-504 are performed at a fixed distance, and a first correspondence between a different blood flow rate and thickness is determined. For the distance, 501-504 are performed at a fixed thickness, and a first correspondence between a different blood flow rate and distance is determined.

For example, when determining the first correspondence between the different blood flow rate and the distance GAP, in 501, at a preset blood flow rate V1 and thickness parameter T, a plurality (M) of blood flow magnetic resonance signals corresponding to different distances are calculated using Maxwell's equations. Each blood flow magnetic resonance signal reflects the blood flow magnetic resonance signal strength at a different slice position. With regard to implementations of Maxwell's equations, reference may be made to the prior art. By means of substituting the preset blood flow rate V1 and thickness parameter T into Maxwell's equations, as well as a preset scan sequence (or scan parameter), the relationship between the blood flow magnetic resonance signal strength and the scanning slice position and the distance can be obtained.

FIG. 6 is a schematic diagram of a plurality of blood flow magnetic resonance signals according to embodiments of the present application. As shown in FIG. 6, the blood flow rate V1 is preset to 5 cm/s, and the thickness parameter T=40 mm. When GAP is equal to 0, for the slice positions 1, 2, 3, 4, 5, 6, 7, 8, 9, 10, 11 and 12, and given the preset scan sequence, a blood flow magnetic resonance signal S1(t) corresponding to GAP=0 is calculated by means of Maxwell's equations. When GAP is equal to 2, for the slice positions 1, 2, 3, 4, 5, 6, 7, 8, 9, 10, 11 and 12, and given the preset scan sequence, a blood flow magnetic resonance signal S2(t) corresponding to GAP=2 is calculated by means of Maxwell's equations. When GAP is equal to 4, for the slice positions 1, 2, 3, 4, 5, 6, 7, 8, 9, 10, 11 and 12, and given the preset scan sequence, a blood flow magnetic resonance signal S3(t) corresponding to GAP=4 is calculated by means of Maxwell's equations, and so on, until the blood flow magnetic resonance signals S4(t), S5(t), S6(t), S7(t), S8(t), S9(t), and S10(t) corresponding to GAP=4, 6, 8, 10, 12, 14, 16, 18 and 20 are obtained. Here, the number M is, for example, 10, but the embodiments of the present application are not limited thereto. In 502, a blood flow magnetic resonance signal having the lowest signal strength among the plurality of blood flow magnetic resonance signals is determined. As shown in FIG. 6, when GAP=0, the strength of the corresponding blood flow magnetic resonance signal S1(t) is the lowest. In 503, the distance corresponding to the blood flow magnetic resonance having the lowest signal strength is taken as a reference distance. As shown in FIG. 6, GAP=0 is taken as the reference distance. In 504, a first relational function between the blood flow rate and the distance is determined according to the preset blood flow rate and the reference distance.

It can be seen from the above content that, given the preset scan sequence and at the blood flow rate V1=5 cm/s and the thickness parameter T, when GAP=0, the blood flow magnetic resonance signal is the smallest, i.e., the artifact signal in the magnetic resonance image due to blood flow is the smallest. That is, given the preset scan sequence and at the blood flow rate V1=5 cm/s and the thickness parameter T, the optimal GAP parameter value is 0. The first relational function y1=f(x) between the blood flow rate and the distance can be derived according to Maxwell's equations, where x represents the blood flow rate, and y1 represents the distance. The preset blood flow rate V1=5 cm/s and the corresponding reference distance of 0 are substituted into the first relational function, then exact values of various coefficients in the first relational function can be obtained, that is, the specific form of the first relational function between the blood flow rate and the distance can be obtained. For any x, a corresponding y1 can be calculated.

For example, in determining the first correspondence between the different blood flow rate and the thickness, in 501, at a preset blood flow rate V2 and distance parameter D, a plurality (N) of blood flow magnetic resonance signals corresponding to different distances are calculated using Maxwell's equations. Each blood flow magnetic resonance signal reflects the blood flow magnetic resonance signal strength at a different slice position. With regard to implementations of Maxwell's equations, reference may be made to the prior art. By means of substituting the preset blood flow rate V2 and distance parameter D into Maxwell's equations, as well as a preset scan sequence, the relationship between the blood flow magnetic resonance signal strength and the scanning slice position and the distance can be obtained.

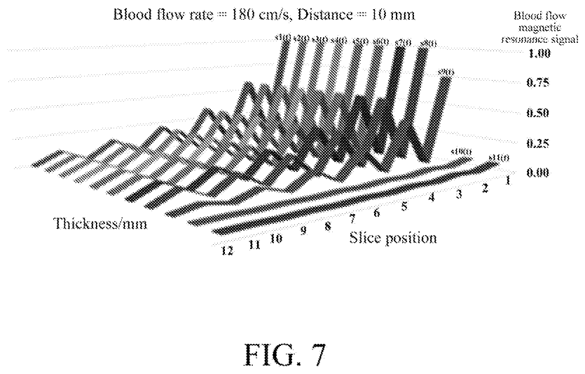

FIG. 7 is a schematic diagram of a plurality of blood flow magnetic resonance signals according to embodiments of the present application. As shown in FIG. 7, the blood flow rate V2 is preset to 180 cm/s, and the distance parameter D=10 mm. When the thickness is equal to 140 mm, for the slice positions 1, 2, 3, 4, 5, 6, 7, 8, 9, 10, 11 and 12, and given the preset scan sequence, a blood flow magnetic resonance signal S1(t) corresponding to T=140 is calculated by means of Maxwell's equations. When T=150, for the slice positions 1, 2, 3, 4, 5, 6, 7, 8, 9, 10, 11 and 12, and given the preset scan sequence, a blood flow magnetic resonance signal S2(t) corresponding to T=150 is calculated by means of Maxwell's equations, and so on, until blood flow magnetic resonance signals S4(t), S5(t), S6(t), S7(t), S8(t), S9(t), S10(t) and S11(t) corresponding to T=160, 170, 180, 190, 200, 210, 220, 230 and 240 are obtained. Here, the number N is, for example, 11, but the embodiments of the present application are not limited thereto. In 502, a blood flow magnetic resonance signal having the lowest signal strength among the plurality of blood flow magnetic resonance signals is determined. As shown in FIG. 7, when T=240, the strength of the corresponding blood flow magnetic resonance signal S11(t) is the lowest. In 503, the thickness corresponding to the blood flow magnetic resonance having the lowest signal strength is taken as a reference thickness. As shown in FIG. 7, T=240 is taken as the reference thickness. In 504, a first relational function between the blood flow rate and the thickness is determined according to the preset blood flow rate and the reference thickness.

It can be seen from the above content that, given the preset scan sequence and at the blood flow rate V2=180 cm/s and the distance parameter D, when T=240, the blood flow magnetic resonance signal is the smallest, i.e., the artifact signal generated in the magnetic resonance image due to blood flow is the smallest. That is, given the preset scan sequence and at the blood flow rate V2=180 cm/s and the distance parameter D, the optimal thickness parameter value is 240 mm. The first relational function y2=f(x) between the blood flow rate and the thickness can be derived according to Maxwell's equations, where x represents the blood flow rate, and y2 represents the thickness. The preset blood flow rate V2=180 cm/s and the corresponding reference thickness of 240 are substituted into the first relational function, then exact values of various coefficients in the first relational function can be obtained, that is, the specific form of the first relational function between the blood flow rate and the thickness can be obtained. For any x, a corresponding y2 can be calculated.

In the above example, the magnetic resonance signals and the first relational function are calculated according to Maxwell's equations, whereby the aforementioned first relational function may be obtained without performing a real scan, which saves scanning time and costs. However, the embodiments of the present application are not limited thereto. For example, the magnetic resonance signals can also be obtained by means of actual scanning, and by changing V1 or V2, a database of multiple sets of blood flow rates and reference spatial saturation band parameters is acquired as the first correspondence, or, multiple sets of corresponding blood flow rates and reference spatial saturation band parameters are acquired and fitted to then obtain the first correspondence, and the like, examples of which are no longer listed herein one by one.

In some embodiments, according to the foregoing second correspondence, the blood flow rate corresponding to the site to be examined can be determined by means of look-up; for example, at least one of the arterial blood flow rate and the venous blood flow rate corresponding to the site to be examined is determined. And, according to the first correspondence, a first spatial saturation band parameter corresponding to the blood flow rate of the site to be examined is determined, which includes performing at least one of the following steps: determining, according to the first correspondence, a venous first spatial saturation band parameter corresponding to the venous blood flow rate of the site to be examined; and determining, according to the first correspondence, an arterial first spatial saturation band parameter corresponding to the arterial blood flow rate of the site to be examined. That is, the blood flow rate (venous or arterial) of the site to be examined is substituted into the first relational function, and a corresponding first spatial saturation band parameter is calculated.

For example, the venous blood flow rate V3 of the site to be examined is substituted into y1=f(x) to obtain the distance y1ᵥ₃, and substituted into y2=f(x) to obtain the thickness y2ᵥ₃, and the arterial blood flow rate V4 of the site to be examined is substituted into y1=f(x) to obtain the distance y1ᵥ₄, and substituted into y2=f(x) to obtain the thickness y2ᵥ₄. That is, a saturation band is added on both sides of a scanning layer or group of layers of the site to be examined, respectively, where the saturation band on one side has a thickness of y2ᵥ₃ and a distance of y1ᵥ₃, and the saturation band on the other side has a thickness of y2ᵥ₄ and a distance of y1ᵥ₄. The above uses adding two saturation bands as an example, but the embodiments of the present application are not limited thereto; for example, only one saturation band may be added. For example, for the abdomen, the optimal spatial saturation band thickness corresponding to the venous blood flow rate is 30 mm, and GAP=5 mm, and the optimal spatial saturation band thickness corresponding to the arterial blood flow rate is 160 mm, and GAP=20 mm.

In some embodiments, according to different sites to be examined, it is determined to select to determine one or both of the arterial blood flow rate and the venous blood flow rate. In other words, according to different sites to be examined, it is determined whether to add a saturation band on one or both sides of the site to be examined (the scanning layer or group of layers). For example, when the site to be examined is a terminal examined site (a palm) of the body, only its corresponding arterial blood flow rate may be determined, or rather, it is determined to add a saturation band on one side of the site to be examined (the scanning layer or group of layers), i.e., the arterial first spatial saturation band parameter is determined. When the site to be examined is a carotid artery vessel, in order to suppress the artifacts generated due to blood flow in a jugular vein (saturate off the venous blood flow signal), only its corresponding venous blood flow rate may be determined, or rather, it is determined to add a saturation band on one side of the site to be examined (the scanning layer or group of layers), i.e., the venous first spatial saturation band parameter is determined. In other examined sites (the abdomen, the liver, etc.), only their corresponding venous blood flow rates and arterial blood flow rates may be determined, or rather, it is determined to add a saturation band on both sides of the site to be examined (the scanning layer or group of layers), i.e., the saturation bands on both sides are determined by the venous first spatial saturation band parameter and the arterial first spatial saturation band parameter, respectively.

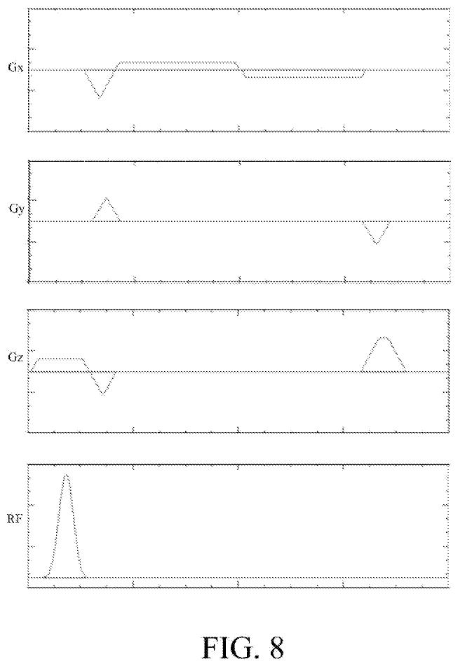

In some embodiments, the scan sequence for scanning the site to be examined is related to the first spatial saturation band parameter. The scan sequence includes a first pulse sequence and a second pulse sequence. After the scanning layer or group of layers of the site to be examined is excited using the second pulse sequence, K-space data of a plurality of layers is acquired. The second pulse sequence is identical to the foregoing preset scan sequence. The second pulse sequence includes a spin echo sequence, a fast spin echo sequence, a gradient echo sequence, a planar echo sequence, a double-echo sequence, an angiographic sequence, and the like. The embodiments of the present application are not limited thereto, and for details, reference may be made to the related art, which are no longer repeated herein one by one. For example, the second pulse sequence includes a radio-frequency pulse sequence and a gradient pulse sequence. FIG. 8 is a schematic diagram when the second pulse sequence is a double-echo sequence according to embodiments of the present application. As shown in FIG. 8, the second pulse sequence includes the gradient pulse Gz for layer selection, gradient pulse Gx for phase encoding and gradient pulse Gy for frequency encoding, and the radio-frequency pulse described above.

In some embodiments, a saturation band is provided on one or both sides of the scanning layer or group of layers, and a first pulse sequence is applied to the saturation band, the first pulse sequence including gradient pulses (e.g., the foregoing gradient pulses in Gx, Gy, and Gz directions) and a radio-frequency pulse, as previously described. The thickness (Thickness) of the saturation band determines the gradient size of a gradient pulse in the first pulse sequence, the gradient pulse referring to a gradient pulse (i.e., Gz) which is applied along with (simultaneously with) the radio-frequency pulse. That is, the thickness of the saturation band determines the size of the Gz gradient in the first pulse sequence, and the distance (GAP) determines the center frequency of the radio-frequency pulse in the first pulse sequence. In addition, when a saturation band is provided on both sides of the scanning layer, two radio-frequency pulses (of which center frequencies correspond to a venous spatial saturation band GAP and an arterial spatial saturation band GAP, respectively) may be included in the first pulse sequence. Two gradient pulses (Gz) applied along with the two radio-frequency pulses differ in magnitude (correspond to a venous spatial saturation band thickness and an arterial spatial saturation band thickness, respectively). When a saturation band is provided on one side of the scanning layer, one radio-frequency pulse may be included in the first pulse sequence.

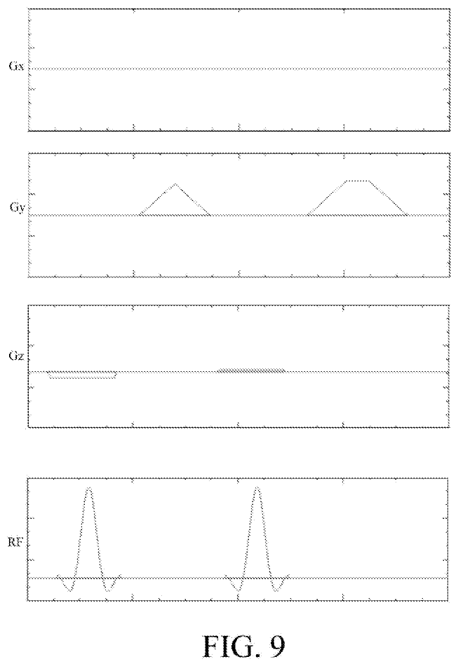

FIG. 9 is a schematic diagram of a first pulse sequence according to embodiments of the present application. As shown in FIG. 9, the first pulse sequence includes two radio-frequency pulses, two gradient pulses (Gz) applied along with the two radio-frequency pulses (the gradients of the two gradient sequences Gz differ in size, and correspond to different saturation band thicknesses, respectively), and a gradient pulse (Gy).

In some embodiments, the first pulse sequence and the second pulse sequence may be applied separately, i.e., the first pulse sequence may be applied before the second pulse sequence, or after the second pulse sequence, or the first pulse sequence and the second pulse sequence may be cross-applied, i.e., merged into a third pulse sequence and then applied. The embodiments of the present application are not limited thereto. For example, when the second pulse sequence is a gradient echo sequence, the first pulse sequence may be applied separately from the second pulse sequence. When the second pulse sequence is FSE, the first pulse sequence may be merged with the FSE to form a third pulse sequence and then applied. These are merely example illustrations, but not limitations.

In some embodiments, the first pulse sequence and the second pulse sequence may be applied according to an application period parameter (which also may be taken as a set scan parameter). For example, if the application period parameter is P, then it means that after every P second pulse sequences are applied, one first pulse sequence may be applied, or rather, when merged into a third pulse sequence and then applied, after every P second pulse sequences, one first pulse sequence may be inserted. The embodiments of the present application are not limited thereto. Examples are no longer listed herein one by one.

In some embodiments, in 202, the site to be examined is scanned using the scan sequence, K-space data is acquired, and the magnetic resonance image of the site to be examined is reconstructed using the K-space data. With regard to how to acquire the K-space data and reconstruct the magnetic resonance image, reference may be made to the prior art, which will not be described herein again.

By means of the aforementioned embodiments, the corresponding spatial saturation band parameter is automatically determined according to the blood flow rate of the site to be examined, and the site to be examined is scanned using the scan sequence related to the first spatial saturation band parameter, to acquire the magnetic resonance image of the site to be examined. As such, artifact signals generated in the magnetic resonance image due to blood flow may be better suppressed, making the magnetic resonance imaging clearer, and the workload of a user can be reduced, improving work efficiency.

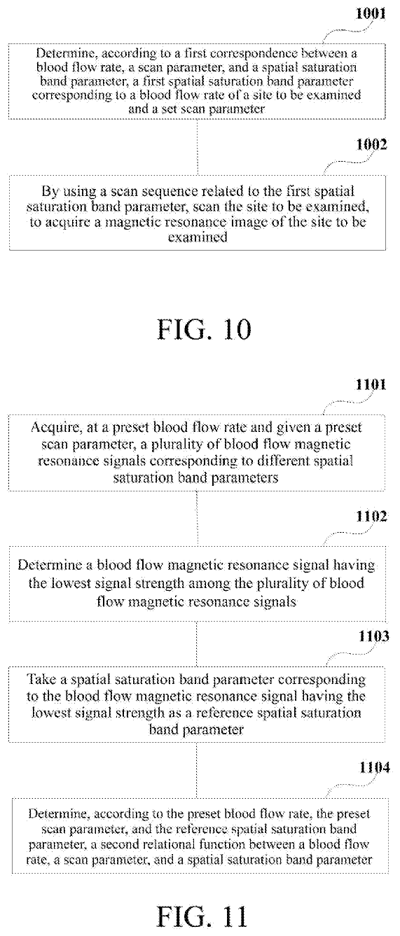

The embodiments of the present application further provide a magnetic resonance scanning and imaging method. FIG. 10 is a schematic diagram of the magnetic resonance scanning and imaging method according to the embodiments of the present application.

As shown in FIG. 10, the method includes: at step 1001, determining, according to a first correspondence between a blood flow rate, a scan parameter, and a spatial saturation band parameter, a first spatial saturation band parameter corresponding to a blood flow rate of a site to be examined and a set scan parameter.

At step 1002, the method includes by using a scan sequence related to the first spatial saturation band parameter, scanning the site to be examined, to acquire a magnetic resonance image of the site to be examined.

Similarities between 1001-1002 and the foregoing embodiments will not be described again. The difference thereof is in that in the first correspondence, a scan parameter needs to be considered in addition to the blood flow rate.

FIG. 11 is a schematic diagram for determining the first correspondence according to embodiments of the present application. As shown in FIG. 11, the method includes: at step 1101, acquiring, at a preset blood flow rate and given a preset scan parameter, a plurality of blood flow magnetic resonance signals corresponding to different spatial saturation band parameters.

At step 1102, the method includes determining a blood flow magnetic resonance signal having the lowest signal strength among the plurality of blood flow magnetic resonance signals.

At step 1103, a spatial saturation band parameter is taken corresponding to the blood flow magnetic resonance signal having the lowest signal strength as a reference spatial saturation band parameter.

Further the method includes at step 1104, determining, according to the preset blood flow rate, the preset scan parameter, and the reference spatial saturation band parameter, a second relational function between the blood flow rate, the scan parameter, and the spatial saturation band parameter.

In some embodiments, 1101-1104 are each performed for different types of spatial saturation band parameters. For example, for the thickness, 1101-1104 are performed at a fixed distance, and a first correspondence between a different blood flow rate, scan parameter, and thickness is determined. For the distance, 1101-1104 are performed at a fixed thickness, and a first correspondence between a different blood flow rate, scan parameter, and distance is determined.

In some embodiments, the scan parameter includes at least one among repetition time, flip angle, the number of scanning layers, and a scanning layer thickness.

For example, in determining the first correspondence between the different blood flow rate, scan parameter and distance GAP, in 1101, at a preset blood flow rate V1 and given a preset scan parameter B1 (B1 including a value of one or more scan parameters) and a thickness parameter T, a plurality of blood flow magnetic resonance signals corresponding to different distances are calculated using Maxwell's equations. Each blood flow magnetic resonance signal reflects the blood flow magnetic resonance signal strength at a different slice position. With regard to implementations of Maxwell's equations, reference may be made to the prior art. By substituting the preset blood flow rate V1, the preset scan parameter B1 and the thickness parameter T into Maxwell's equations, the relationship between the blood flow magnetic resonance signal strength and the scanning slice position and the distance can be obtained. In 1102, a blood flow magnetic resonance signal having the lowest signal strength among the plurality of blood flow magnetic resonance signals is determined. In 1103, the distance corresponding to the blood flow magnetic resonance having the lowest signal strength is taken as a reference distance. In 1104, a second relational function between the blood flow rate, the scan parameter and the distance is determined according to the preset blood flow rate, the preset scan parameter, and the reference distance. It can be seen from the above content that, given the preset scan parameter B1 and at the preset blood flow rate V1=5 cm/s and the thickness parameter T, an optimal GAP parameter value may be determined. The second relational function z1=g(x, b) between the blood flow rate, the scan parameter, and the distance can be derived according to Maxwell's equations, where x represents the blood flow rate, b represents the scan parameter (which may include a repetition time b1, a flip angle b2, a number of scanning layers b3, a scanning layer thickness b4, etc.), and z1 represents the distance. The preset blood flow rate V1=5 cm/s, the preset scan parameter B1 and the corresponding reference distance are substituted into the second relational function, then exact values of various coefficients in the second relational function can be obtained, that is, the specific form of the second relational function between the blood flow rate, the scan parameter, and the distance can be obtained. For any x and b, a corresponding z1 can be calculated.

For example, when determining the first correspondence between the different blood flow rate, scan parameter, and thickness, in 1101, at a preset blood flow rate V2 and given a preset scan parameter B1 and a distance parameter D, a plurality of blood flow magnetic resonance signals corresponding to different distances are calculated using Maxwell's equations. Each blood flow magnetic resonance signal reflects the blood flow magnetic resonance signal strength at a different slice position. With regard to implementations of Maxwell's equations, reference may be made to the prior art. By substituting the preset blood flow rate V2, the preset scan parameter B1, and the distance parameter D into Maxwell's equations, the relationship between the blood flow magnetic resonance signal strength and the scanning slice position and the thickness can be obtained. In 1102, a blood flow magnetic resonance signal having the lowest signal strength among the plurality of blood flow magnetic resonance signals is determined. In 1103, the distance corresponding to the blood flow magnetic resonance signal having the lowest signal strength is taken as a reference thickness. In 1104, a second relational function between the blood flow rate, the scan parameter, and the thickness is determined according to the preset blood flow rate, the preset scan parameter, and the reference thickness. It can be seen from the above content that, given the preset scan parameter BI and at the preset blood flow rate V1=5 cm/s and the distance parameter D, an optimal thickness parameter value may be determined. The second relational function z2=g(x, b) between the blood flow rate, the scan parameter, and the thickness can be derived according to Maxwell's equations, where x represents the blood flow rate, b represents the scan parameter (which may include a repetition time b1, a flip angle b2, a number of scanning layers b3, a scanning layer thickness b4, etc.), and z2 represents the thickness. The preset blood flow rate V1=5 cm/s, the preset scan parameter B1, and the corresponding reference thickness are substituted into the second relational function, then exact values of various coefficients in the second relational function can be obtained, that is, the specific form of the second relational function between the blood flow rate, the scan parameter and the thickness can be obtained. For any x and b, a corresponding z2 can be calculated.

In the above example, the magnetic resonance signals and the second relational function are calculated according to Maxwell's equations, whereby the aforementioned second relational function may be obtained without performing a real scan, which saves scanning time and costs. However, the embodiments of the present application are not limited thereto. For example, the magnetic resonance signals can also be obtained by means of actual scanning, and by changing V1 or V2 and the scan parameter, a database of multiple sets of blood flow rates, scan parameters, and reference spatial saturation band parameters is acquired as the first correspondence, or, multiple sets of corresponding blood flow rates, scan parameters, and reference spatial saturation band parameters are acquired and fitted to then obtain the second correspondence, and the like, examples of which are no longer listed herein one by one.

In some embodiments, according to the foregoing second correspondence, the blood flow rate corresponding to the site to be examined can be determined by means of look-up; for example, at least one of the arterial blood flow rate and the venous blood flow rate corresponding to the site to be examined is determined. And, at least one of the following steps is performed: determining, according to the first correspondence, a venous first spatial saturation band parameter corresponding to the set scan parameter and the venous blood flow rate; and determining, according to the first correspondence, an arterial first spatial saturation band parameter corresponding to the set scan parameter and the arterial blood flow rate. That is, the blood flow rate (venous or arterial) of the site to be examined and the set scan parameter are substituted into the second correspondence, and a corresponding first spatial saturation band parameter is calculated.

For example, the venous blood flow rate V3 of the site to be examined and the set scan parameter B2 are substituted into z1=g(x, b) to obtain the distance z1ᵥ₃, and substituted into z2=g(x, b) to obtain the thickness z2ᵥ₃, and the arterial blood flow rate V4 of the site to be examined and the set scan parameter B2 are substituted into z1=g(x, b) to obtain the distance z1ᵥ₄, and into z2=g(x, b) to obtain the thickness z2ᵥ₄. That is, a saturation band is added on both sides of a scanning layer or group of layers of the site to be examined, respectively, where the saturation band on one side has a thickness of z2ᵥ₃ and a distance of z1ᵥ₃, and the saturation band on the other side has a thickness of z2ᵥ₄ and a distance of z1ᵥ₄. The above uses adding two saturation bands as an example, but the embodiments of the present application are not limited thereto, for example, only one saturation band may be added. Reference may be made to the foregoing embodiments for specifically how to add a saturation band, which will not be repeated here.

In the foregoing embodiments, for each site to be examined, the scan parameter is fixed when determining the first correspondence and during the actual scan. That is, for each site to be examined, the second pulse sequence is the same as the preset scan sequence (for different sites to be examined, the applied second pulse sequence (the preset scan sequence) may be different). This differs from the foregoing embodiments in that, in the present embodiment, because the scan parameter is considered in addition to the blood flow rate when determining the first correspondence, for each site to be examined, the scan parameter is variable when determining the first correspondence and during the actual scan. That is, for each site to be examined, the second pulse sequence may be different from the preset scan sequence. Therefore, the scan sequence for scanning the site to be examined is related to the first spatial saturation band parameter and the set scan parameter. The scan sequence includes a first pulse sequence and a second pulse sequence. After the scanning layer or group of layers of the site to be examined is excited using the second pulse sequence, K-space data of a plurality of layers is acquired. The second pulse sequence is related to the foregoing set scan parameter B2 (which includes a repetition time, a flip angle, a number of scanning layers, a scanning layer thickness, etc.). The set scan parameter B2 may be the same as or different from the preset scan parameter B1, and the embodiments of the present application are not limited thereto. The second pulse sequence includes a spin echo sequence, a fast spin echo sequence, a gradient echo sequence, a planar echo sequence, a double-echo sequence, an angiographic sequence, and the like. The embodiments of the present application are not limited thereto. Reference may be made to the related art for specifically how to determine the second pulse sequence according to the scan parameter B2. For example, the scanning layer thickness determines the gradient size of a gradient pulse in the second pulse sequence, and the flip angle determines the flip angle of a radio-frequency pulse in the second pulse sequence, and so on, which will not be repeated herein one by one. With regard to the first pulse sequence, reference may be made to the foregoing embodiments, which will not be repeated here.

Other implementations, etc., related to the scan sequence, scanning, imaging, are the same as the foregoing embodiments, which will not be repeated here.

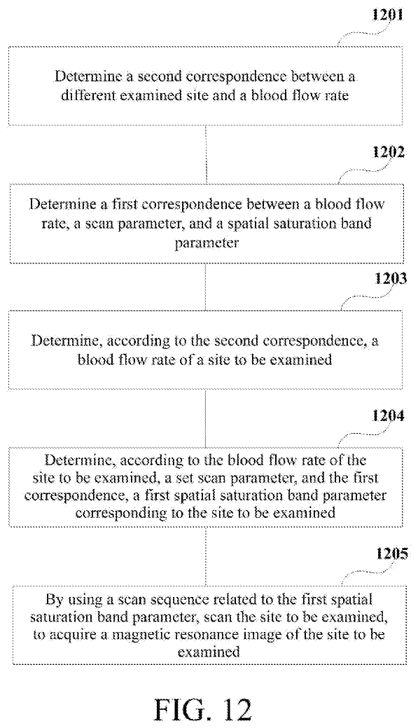

FIG. 12 is a schematic diagram of a magnetic resonance scanning and imaging method according to embodiments of the present application. As shown in FIG. 12, the method includes: determining a second correspondence between a different examined site and a blood flow rate at step.

At step 1202, a first correspondence is determined between a blood flow rate, a scan parameter, and a spatial saturation band parameter.

At step 1203, a blood flow rate of a site to be examined is determined according to the second correspondence.

At step 1204, the method includes determining, according to the blood flow rate of the site to be examined, a set scan parameter, and the first correspondence, a first spatial saturation band parameter corresponding to the site to be examined.

At step 1205, using a scan sequence related to the first spatial saturation band parameter, the site to be examined is scanned, to acquire a magnetic resonance image of the site to be examined.

Implementations of 1201-1205 are as described previously, and will not be repeated here.

It should be noted that FIG. 2, FIG. 5, FIG. 10, FIG. 11, and FIG. 12 above merely schematically illustrate the embodiments of the present application, but the present application is not limited thereto. For example, the order of execution between operations may be appropriately adjusted. In addition, some other operations may be added or some operations may be omitted. Those skilled in the art could make appropriate variations according to the above content, rather than being limited by the disclosures of FIG. 2, FIG. 5, FIG. 10, FIG. 11, and FIG. 12 described above.

The above embodiments merely provide illustrative descriptions of the embodiments of the present application. However, the present application is not limited thereto, and appropriate variations may be made on the basis of the above embodiments. For example, each of the above embodiments may be used independently, or one or more among the above embodiments may be combined.

By means of the aforementioned embodiments, the corresponding spatial saturation band parameter is automatically determined according to the blood flow rate of the site to be examined and the set scan parameter, and the site to be examined is scanned using the scan sequence related to the first spatial saturation band parameter and the set scan parameter, to acquire the magnetic resonance image of the site to be examined. As such, artifact signals generated in the magnetic resonance image due to blood flow may be better suppressed, making the magnetic resonance imaging clearer, and the workload of a user can be reduced, improving work efficiency.

An embodiment of the present application further provides a magnetic resonance imaging system. The configuration of the magnetic resonance imaging system is as shown in FIG. 1, and similarities are not repeated here.

In some embodiments, the difference from the foregoing magnetic resonance imaging system in FIG. 1 is that the controller 130 is further used to control to determine, according to a first correspondence between a blood flow rate and a spatial saturation band parameter, a first spatial saturation band parameter corresponding to a blood flow rate of a site to be examined, or to determine, according to a first correspondence between a blood flow rate, a scan parameter and a spatial saturation band parameter, a first spatial saturation band parameter corresponding to the blood flow rate of the site to be examined and a set scan parameter; and control the scanning unit 110 to use a scan sequence related to the first spatial saturation band parameter to scan the site to be examined, to acquire a magnetic resonance image of the site to be examined.

In some embodiments, for implementations of the controller 130, reference may be made to the magnetic resonance scanning and imaging method according to the foregoing embodiments. The functions of the controller 130 and the processor 150 may be integrated into one chip, or implemented by separate chips, and the embodiments of the present application are not limited thereto.

In some embodiments, the controller 130 includes a computer processor and a storage medium. Recorded on storage medium is a program for predetermined data processing to be executed by the computer processor. For example, stored on the storage medium may be a program for performing scan processing (e.g., including waveform design/conversion, etc.), image reconstruction, image processing, etc. For example, stored on the storage medium may be a program for implementing the method for determining a spatial saturation band parameter according to the embodiments of the present invention. The method for determining a spatial saturation band parameter includes: determining a first correspondence between a blood flow rate and a spatial saturation band parameter, and determining, according to the first correspondence, a first spatial saturation band parameter corresponding to a blood flow rate of a site to be examined; or determining a first correspondence between a blood flow rate, a scan parameter, and a spatial saturation band parameter, and determining, according to the first correspondence, a first spatial saturation band parameter corresponding to the blood flow rate of the site to be examined and a set scan parameter. The aforementioned storage medium may include, for example, a ROM, a floppy disk, a hard disk, an optical disk, a magnetic disk, a CD-ROM, or a non-volatile memory card.

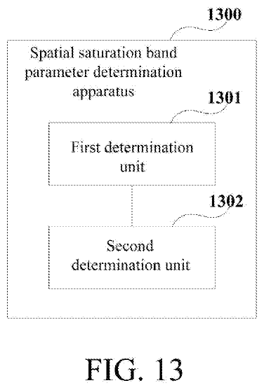

The embodiments of the present application further provide an apparatus for determining a spatial saturation band parameter. FIG. 13 is a schematic diagram of an apparatus for determining a spatial saturation band parameter according to embodiments of the present invention.

As shown in FIG. 13, the apparatus includes: a first determination unit 1301, that determines a first correspondence between a blood flow rate and a spatial saturation band parameter.

The apparatus further includes a second determination unit 1302, that determines, according to the first correspondence, a first spatial saturation band parameter corresponding to a blood flow rate of a site to be examined.

In some embodiments, the first determination unit 1301 may further determine a first correspondence between a blood flow rate, a scan parameter, and a spatial saturation band parameter; and the second determination unit 1302 determines, according to the first correspondence, a first spatial saturation band parameter corresponding to the blood flow rate of the site to be examined and a set scan parameter.

The apparatus may further include (not shown): a third determination unit, that determines a second correspondence between a different examined site and a blood flow rate. The second determination unit 1302 determines, according to the second correspondence, the blood flow rate corresponding to the site to be examined.

Implementations of the first determination unit 1301, the second determination unit 1302, and the third determination unit are as described above, and are not repeated here.

The embodiments of the present application further provide a computer-readable program, which, when executed in an apparatus or an MRI system, causes a computer to perform, in the apparatus or the MRI system, the magnetic resonance scanning and imaging method according to the foregoing embodiments.

The embodiments of the present invention further provide a storage medium having a computer-readable program stored therein, the computer-readable program causing a computer to perform, in an apparatus or an MRI system, the magnetic resonance scanning and imaging method according to the foregoing embodiments.

The above apparatus and method of the present application can be implemented by hardware, or can be implemented by hardware in combination with software. The present application relates to the foregoing type of computer-readable program. When executed by a logic component, the program causes the logic component to implement the foregoing apparatus or a constituent component, or causes the logic component to implement various methods or steps as described above. The present application further relates to a storage medium for storing the above program, such as a hard disk, a disk, an optical disk, a DVD, a flash memory, etc.

The method/apparatus described in view of the embodiments of the present application may be directly embodied as hardware, a software module executed by a processor, or a combination of the two. For example, one or more of the functional block diagrams and/or one or more combinations of the functional block diagrams shown in the drawings may correspond to either respective software modules or respective hardware modules of a computer program flow. The foregoing software modules may respectively correspond to the steps shown in the figures. The foregoing hardware modules can be implemented, for example, by firming the software modules using a field-programmable gate array (FPGA).

The software modules may be located in a RAM, a flash memory, a ROM, an EPROM, an EEPROM, a register, a hard disk, a portable storage disk, a CD-ROM, or any other form of storage medium known in the art. The storage medium may be coupled to a processor, so that the processor can read information from the storage medium and can write information into the storage medium. Alternatively, the storage medium may be a component of the processor. The processor and the storage medium may be located in an ASIC. The software module may be stored in a memory of a mobile terminal, and may also be stored in a memory card that can be inserted into a mobile terminal. For example, if a device (such as a mobile terminal) uses a large-capacity MEGA-SIM card or a large-capacity flash memory device, the software modules can be stored in the MEGA-SIM card or the large-capacity flash memory apparatus.

One or more of the functional blocks and/or one or more combinations of the functional blocks shown in the accompanying drawings may be implemented as a general-purpose processor, a digital signal processor (DSP), an application-specific integrated circuit (ASIC), a field-programmable gate array (FPGA) or other programmable logic devices, discrete gate or transistor logic devices, a discrete hardware assembly, or any appropriate combination thereof for implementing the functions described in the present application. The one or more functional blocks and/or the one or more combinations of the functional blocks shown in the accompanying drawings may also be implemented as a combination of computing devices, such as a combination of a DSP and a microprocessor, multiple microprocessors, one or more microprocessors in communication combination with a DSP, or any other such configuration.

The present application is described above with reference to specific embodiments. However, it should be clear to those skilled in the art that the foregoing description is merely illustrative and is not intended to limit the scope of protection of the present application. Various variations and modifications may be made by those skilled in the art according to the principle of the present application, and said variations and modifications also fall within the scope of the present application.

What is claimed is:

1. A magnetic resonance scanning and imaging method, characterized in that the method comprises:
determining, according to a first correspondence between a blood flow rate and a spatial saturation band parameter, a first spatial saturation band parameter corresponding to a blood flow rate of a site to be examined; and
by using a scan sequence related to the first spatial saturation band parameter, scanning the site to be examined, to acquire a magnetic resonance image of the site to be examined.

2. The method according to claim 1 further comprising:
determining a second correspondence between a different examined site and a blood flow rate; and
determining, according to the second correspondence, the blood flow rate of the site to be examined.

3. The method according to claim 1, wherein the blood flow rate comprises at least one among a venous blood flow rate and an arterial blood flow rate.

4. The method according to claim 1, wherein the method further comprises:
determining the first correspondence, comprising:
acquiring, at a preset blood flow rate, a plurality of blood flow magnetic resonance signals corresponding to different spatial saturation band parameters;
determining a blood flow magnetic resonance signal having the lowest signal strength among the plurality of blood flow magnetic resonance signals;
taking a spatial saturation band parameter corresponding to the blood flow magnetic resonance signal having the lowest signal strength as a reference spatial saturation band parameter; and
determining, according to the preset blood flow rate and the reference spatial saturation band parameter, a first relational function between blood flow rate and a spatial saturation band parameter.

5. The method according to claim 4, wherein acquiring, at a preset blood flow rate, a plurality of blood flow magnetic resonance signals corresponding to different spatial saturation band parameters comprises:
substituting the preset blood flow rate and a spatial saturation band parameter into Maxwell's equations, and calculating to obtain a blood flow magnetic resonance signal corresponding to the spatial saturation band parameter.

6. The method according to claim 1, wherein the step of determining, according to a first correspondence between a blood flow rate and a spatial saturation band parameter, a first spatial saturation band parameter corresponding to a blood flow rate of a site to be examined comprises:
determining at least one among a venous blood flow rate and an arterial blood flow rate corresponding to the site to be examined; and performing at least one of the following steps:
determining, according to the first correspondence, a venous first spatial saturation band parameter corresponding to the venous blood flow rate; and
determining, according to the first correspondence, an arterial first spatial saturation band parameter corresponding to the arterial blood flow rate.

7. The method according to claim 1, wherein determining, according to a first correspondence between a blood flow rate and a spatial saturation band parameter, a first spatial saturation band parameter corresponding to a blood flow rate of a site to be examined comprises:
determining, according to a first correspondence between a blood flow rate, a scan parameter and a spatial saturation band parameter, a first spatial saturation band parameter corresponding to a blood flow rate of a site to be examined and a set scan parameter.

8. The method according to claim 7, wherein the method further comprises:
determining the first correspondence, comprising:
acquiring, at a preset blood flow rate and given a preset scan parameter, a plurality of blood flow magnetic resonance signals corresponding to different spatial saturation band parameters;
determining a blood flow magnetic resonance signal having the lowest signal strength among the plurality of blood flow magnetic resonance signals;
taking a spatial saturation band parameter corresponding to the blood flow magnetic resonance signal having the lowest signal strength as a reference spatial saturation band parameter; and
determining, according to the preset blood flow rate, the preset scan parameter and the reference spatial saturation band parameter, a second relational function between the blood flow rate, the scan parameter, and the spatial saturation band parameter.

9. The method according to claim 8, wherein acquiring, at a preset blood flow rate and given a preset scan parameter, a plurality of blood flow magnetic resonance signals corresponding to different spatial saturation band parameters comprises:
substituting the preset blood flow rate, the preset scan parameter, and a spatial saturation band parameter into Maxwell's equations, and calculating to obtain a blood flow magnetic resonance signal corresponding to the spatial saturation band parameter.

10. The method according to claim 7, wherein the step of determining, according to a first correspondence between a blood flow rate, a scan parameter and a spatial saturation band parameter, a first spatial saturation band parameter corresponding to a blood flow rate of the site to be examined and a set scan parameter comprises:
determining at least one among a venous blood flow rate and an arterial blood flow rate corresponding to the site to be examined; and performing at least one of the following steps:
determining, according to the first correspondence, a venous first spatial saturation band parameter corresponding to the set scan parameter and the venous blood flow rate; and
determining, according to the first correspondence, an arterial first spatial saturation band parameter corresponding to the set scan parameter and the arterial blood flow rate.

11. The method according to claim 1, wherein the spatial saturation band parameter comprises at least one among the distance between the spatial saturation band and the site to be examined, and the thickness of the spatial saturation band.

12. The method according to claim 7, wherein the scan parameter comprises at least one among repetition time, flip angle, the number of scanning layers, and scanning layer thickness.

13. The method according to claim 1, wherein the scan sequence comprises a first pulse sequence, at least one among a gradient size and a center frequency of pulses in the first pulse sequence being related to the first spatial saturation band parameter.

14. The method according to claim 13, wherein the scan sequence further comprises a second pulse sequence related to a set scan parameter.

15. A magnetic resonance imaging system, wherein the system comprises:
a scanning unit; and
a controller used to determine, according to a first correspondence between blood flow rate and spatial saturation band parameters, a first spatial saturation band parameter corresponding to a blood flow rate of a site to be examined; and control the scanning unit to use a scan sequence related to the first spatial saturation band parameter to scan the site to be examined, to acquire a magnetic resonance image of the site to be examined.

16. The system according to claim 15, wherein the controller is further used to determine, according to a first correspondence between a blood flow rate, a scan parameter and a spatial saturation band parameter, a first spatial saturation band parameter corresponding to the blood flow rate of the site to be examined and a set scan parameter.

17. The system according to claim 15, wherein the controller determines, according to a second correspondence between a different examination site and a blood flow rate, the blood flow rate of the site to be examined.

18. The system according to claim 15, wherein the controller determines at least one among a venous blood flow rate and an arterial blood flow rate corresponding to the site to be examined; and performs at least one of the following steps: determining, according to the first correspondence, a venous first spatial saturation band parameter corresponding to the venous blood flow rate; and determining, according to the first correspondence, an arterial first spatial saturation band parameter corresponding to the arterial blood flow rate.

19. The system according to claim 16, wherein the controller determines at least one among a venous blood flow rate and an arterial blood flow rate corresponding to the site to be examined; and performs at least one of the following steps: determining, according to the first correspondence, a venous first spatial saturation band parameter corresponding to the set scan parameter and the venous blood flow rate; and determining, according to the first correspondence, an arterial first spatial saturation band parameter corresponding to the set scan parameter and the arterial blood flow rate.

20. The system according to claim 15, wherein the spatial saturation band parameter comprises at least one among the distance between the spatial saturation band and the site to be examined, and the thickness of the spatial saturation band.

21. The system according to claim 16, wherein the scan parameter comprises at least one among repetition time, flip angle, the number of scanning layers, and scanning layer thickness.

22. The system according to claim 15, wherein the scan sequence comprises a first pulse sequence, at least one among a gradient size and a center frequency of pulses in the first pulse sequence being related to the first spatial saturation band parameter.
